(19) United States
(12) Patent Application Publication
(10) Pub. No.: US 2015/0031980 A1
(43) Pub. Date: Jan. 29, 2015

(54) BRAIN SIGNAL MANAGEMENT SYSTEM AND BRAIN SIGNAL MANAGEMENT METHOD USING THE SAME

(71) Applicant: YBRAIN INC., Seoul (KR)

(72) Inventors: Kyongsik Yun, Seoul (KR); Yongwook Chae, Seoul (KR); Kiwon Lee, Seoul (KR)

(73) Assignee: YBRAIN INC., Seoul (KR)

(21) Appl. No.: 14/056,519

(22) Filed: Oct. 17, 2013

Related U.S. Application Data
(60) Provisional application No. 61/858,856, filed on Jul. 26, 2013.

(51) Int. Cl. A61B 5/00; A61B 5/055; A61B 8/00; A61B 5/0476

(52) U.S. Cl. CPC A61B 5/4064; A61B 5/0476; A61B 5/055; A61B 8/00; A61B 5/0075
USPC 600/410; 600/544; 600/437; 600/473; 600/300

(57) ABSTRACT

A system and method for brain signal management are provided. In some embodiments, a brain signal management system includes a detecting module configured to measure a first signal which indicates a state of a brain, and a controlling module configured to generate the second signal by transforming the first signal into the second signal in a time reversal order.

### CROSS-REFERENCE TO RELATED APPLICATION

This application claims the priority of the U.S. Provisional Patent Application No. 61/858,856, filed on Jul. 26, 2013 in the United States Patent and Trademark Office (USPTO), and all the benefits accruing therefrom under 35 U.S.C. §119, the contents of which in their entirety are herein incorporated by reference.

### TECHNICAL FIELD

The present disclosure relates to a brain signal management system and a method of brain signal management.

### BACKGROUND

The brain, an internal organ in the head of a human body, is the supreme and vital organ of the nervous system, and is composed of the cerebrum, cerebellum, midbrain, pons and medulla oblongata.

Furthermore, the brain generates electroencephalograms, signals measured at the epidermis of the brain, which is the sum of activation levels of neurons.

Measurements of the brain comprise EEG (electroencephalogram) that scans and measures electroencephalograms received from electrodes on a pad installed on the scalp, CT (computed tomography) that scans and measures the brain using radiation or ultrasound to perform a tomographic scan from various viewpoints, and MRI (magnetic resonance imaging) that scans the brain exploiting the property of nuclear magnetic resonance, and so on.

Besides, brain stimulation is used as means to an expected end by stimulating certain parts of the brain using electricity or ultrasound or a magnetic field. The brain stimulation generally consists of invasive brain stimulation and non-invasive brain stimulation.

Invasive brain stimulation is a method that inserts electrodes into the brain by surgery and delivers electric signals, and non-invasive brain stimulation is a method that stimulates the brain not inserting electrodes within the cranium and thus accomplishing an anticipated end.

### SUMMARY

In accordance with some embodiments, there is provided a brain signal management system comprising a detecting module configured to measure a first signal which indicates a state of a brain, and a controlling module configured to generate a second signal by transforming the first signal into the second signal in a time reversal order.

In accordance with some embodiments, there is provided a brain signal management system comprising a controlling module configured to transform a first signal which indicates a state of a brain in a time reversal order and generate a second signal based on the transformed first signal, and a stimulating module configured to send the generated second signal to the brain or another brain.

In accordance with some embodiments, there is provided a method of a brain signal management system performed by a brain signal management system, the method comprising measuring a first signal which indicates a state of a brain and generating a second signal by transforming the first signal in a time reversal order into the second signal.

### BRIEF DESCRIPTION OF THE DRAWINGS

FIG. 1 is a schematic block diagram of a brain signal management system according to some embodiments.

FIG. 2 is a flow chart of a method of brain signal management according to some embodiments.

FIG. 3 is a schematic structure and direction of transmission of a first signal in a brain signal management system according to some embodiments.

FIG. 4 is a schematic structure and direction of transmission of a second signal in a brain signal management system according to some embodiments.

### DETAILED DESCRIPTION

A system and method for brain signal management will be described more fully hereinafter with reference to the accompanying drawings, in which some embodiments are shown. Advantages and features of some embodiments and methods of accomplishing the same are hereafter detailed with reference to the accompanying drawings. The system and method for brain signal management are embodied in different forms and should not be construed as limited to the embodiments set forth herein. Rather, these embodiments are provided so that this disclosure will be thorough and complete, and will fully convey the scope of the system and method for brain signal management to those skilled in the art. The system and method for brain signal management are only defined by the category of the following claims. The same reference numbers indicate the same components throughout the specification.

Unless defined otherwise, all technical and scientific terms used herein have the same meaning as commonly understood by one of ordinary skill in the art to which this application belongs. It is noted that the use of any and all examples, or exemplary terms provided herein is intended merely to better illuminate the system and method for brain signal management and is not a limitation on the scope of the system and method for brain signal management unless otherwise specified. Further, unless defined otherwise, all terms defined in generally used dictionaries may not be overly interpreted.

The use of the terms “a” and “an” and “the” and similar referents in the context of describing the system and method for brain signal management (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

A detailed description of some embodiments of the system and method for brain signal management is hereafter presented with reference to the accompanying drawings.

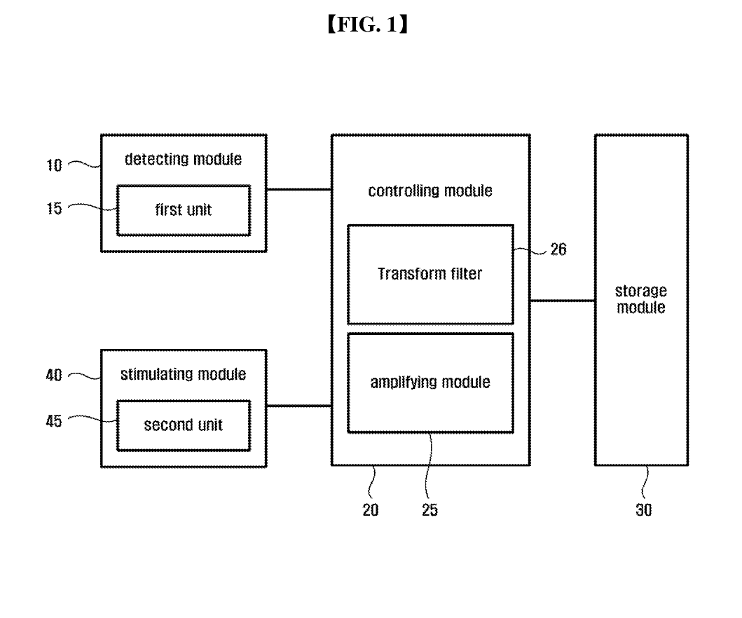

FIG. 1 is a schematic block diagram of a brain signal management system according to some embodiments.

As shown in the drawings, a brain signal management system in accordance with some embodiments includes a detecting module 10, a controlling module 20, a storage module 30 and a stimulating module 40.

The detecting module 10 measures a first signal 3 which indicates a state of a brain, and the first unit 15, in some embodiments, includes a first electrode configured to touch the scalp 1 of a human. That is, the detecting module is composed in a variety of forms to detect signals produced from the brain. For example, the detecting module detects signals from the brain in a number of ways, such as ultrasound and magnetic resonance imaging (MRI). In those cases, the detecting module 10 contains a detecting structure corresponding to each type of signals. Further, in some embodiments, the detecting module 10 is constituted as a form of noncontact module.

In some embodiments, a first signal 3, received from the detecting module 10 having various compositions aforementioned, includes at least one type of the following signals, such as electroencephalograms (EEG), impedance signals, acoustic signals, magnetic signals, mechanical signals, chemical signals, optical signals, ultrasonic signals, and so on. In some embodiments, the first signal 3 is another type of signal other than the signals above. For instance, the optical signals are near-infrared spectroscopy.

Some embodiments where the detecting module 10 contains a first electrode 15 are provided for explanation hereafter.

In some embodiments, the first electrode 15 is made of a conductive material, and the first electrode 15 measures the first signal 3, an electroencephalogram (EEG) of a certain mode. The first electrode 15 is arranged at, but not limited to, a position where the first signal 3 is detected from at least one of the following areas: dorsolateral prefrontal cortex, ventromedial prefrontal cortex, primary motor area, temporal lobe, and occipital lobe.

The controlling module 20, being electrically connected with the detecting module 10, generates a second signal 5 which is a time-reversed signal of the first signal 3 measured via the detecting module 10. The controlling module 20 includes one or more physical, actual storage devices. Examples of physical, actual storage devices include, but are not limited to, magnetic media such as, a hard disk, a floppy disk, and a magnetic tape, optical media such as a CD-ROM and a DVD, magneto-optical media such as a floptical disk, and a hardware device configured especially to store and execute a program, such as a ROM, a RAM, a solid state drive, and a flash memory. The controlling module 20 is implemented by one or more programmed processors and/or application-specific integrated circuits (ASICs).

In the same way as the first signal 3, in some embodiments, the second signal 5 includes at least one of the following types of signals: electroencephalograms, impedance signals, acoustic signals, magnetic signals, mechanical signals, chemical signals, optical signals, or ultrasonic signals. In some embodiments, the second signal 5 is another type of signal, for instance, the optical signals are near-infrared spectroscopy.

The second signal 5 is a time-reversed signal of the first signal 3 measured at the detecting module 10, that is, a reversed image. In the meantime, the first signal 3 and the second signal 5 have different types: for instance, the first signal 3 is an electroencephalographic electric signal, whereas the second signal 5 has a structure of ultrasonic signals. In this case, an intermediate signal transformed in a time-reversal order from the first signal 3 is firstly generated, and then, the intermediate signal is transformed into a different kind from the first signal 3 to obtain the second signal 5, but not limited to this. In some embodiments, the first signal 3 is transformed into the second signal 5 which has a different type from the first signal 3, and then, the transformed second signal 5 is further transformed in a time-reversal order.

In some embodiments, a transformation filter 26 for transforming the first signal 3 as a different kind of signal into the second signal 5 is further included in the controlling module 20. The transformation filter 26 as such performs a mutual transforming of different kinds of signals.

Meanwhile, in some embodiments, the controlling module 20 includes an amplifying module 25 that amplifies the first signal 3. An amplifying module is to transform the first signal 3 measured at the first electrode 15 into a signal easier to observe before transforming the first signal 3 into the second signal 5.

Also, in some embodiments, the controlling module 20 further includes a noise removal module that removes noise from the first signal 3.

In some embodiments, the controlling module 20 receives the first signal 3 and the second signal 5 from an external database other than the detecting module 10, wherein the second signal 5 is transformed in a time reversal order from the first signal 3, thus dealing with signals with no additional transformation process.

The storage module 30 is electrically connected with the controlling module 20, and the storage module 30 stores one or more second signals 5 by distinguishing the one or more second signals 5 in accordance with plural specific states of the one or more second signals 5.

In some embodiments, the storage module 30 separately stores one or more first signals 3, one or more signals before transformed into one or more second signals 5, in accordance with a plurality of specific states of the one or more first signal 5. In some embodiment, in a process of the storage module, a step that produces the one or more second signals 5 is skipped.

Furthermore, a brain signal management system in accordance with some embodiments further comprises a stimulating module 40 that sends the second signal 5 to the brain. The second signal 5 is generated based on data stored in the storage module 30, or directly received from the controlling module 20. The stimulating module 40 includes, but is not limited to, a second unit 45, for instance, a second electrode 45, and in some embodiments, the second electrode 45 is constituted as another form different from the first electrode 15 of the detecting module 10. For instance, in the case where the stimulating module 40 is the second electrode 45, the second electrode 45 includes conductive material, and the second electrode 45 sends the second signal 5 which indicates a particular state of the brain, for example, the signals from the stimulating module 40 is such as electroencephalogram, ultrasonic signals, near-infrared spectroscopy or magnetic signals, to the brain. In some embodiments which are differ from FIG. 1, the first unit 15 and the second unit 45 are formed as one unit, e.g., as a single electrode. In some embodiments, the stimulating module 40 is adhered to the scalp 1, and the stimulating module 40 is be separately arranged from the scalp 1 so that the stimulating module 40 is released from a position where stimulating module 40 is detached from the scalp 1.

In some embodiments, the storage module 30, as the some embodiments aforementioned, directly stores a first signal 3 measured by the detecting module 10, without storing the second signal 5 transformed in a time-reversal order. In these embodiments, the controlling module 20 loads the first signal 3 which indicates a particular state of the brain and is stored in the storage module 30, and the controlling module 20 transforms the loaded first signal 3 into the second signal 5. In addition, the second signal 5 is sent to the brain via the second electrode 45 in the stimulating module 40.

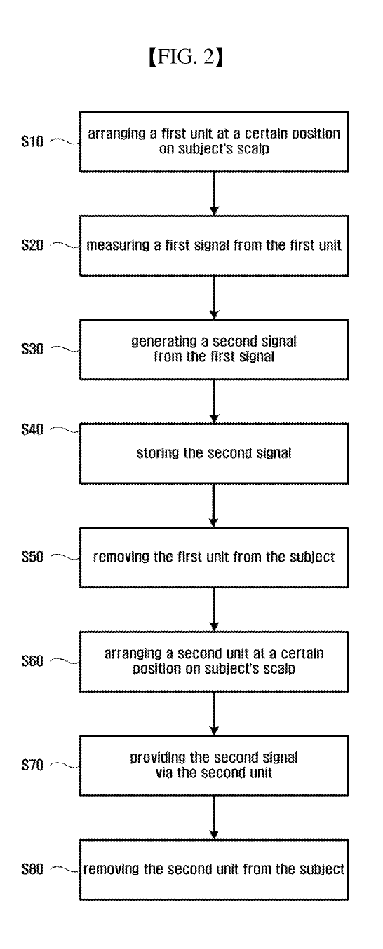

One or more methods to manage electroencephalograms using a brain signal management system complying with the composition above are described with reference to FIGS. 2-4.

The method of brain signal management according to some embodiments comprises the steps of: signal measurement from a brain; and brain stimulation. Hereafter, a method of brain signal management is shown by, but not limited to, a case in which a detecting module 10 and a stimulating module 40 include electrodes delivering or detecting a specific signal, such as near-infrared spectroscopy, ultrasonic signals or magnetic signals as described before. However, in at least one embodiment, the detecting module 10 and the stimulating module 40 include other forms than electrodes.

The step of signal measurement of the method of brain signal management in accordance with some embodiments is as follows: first, when a subject's brain is in a specific state such as excitement or normality, a first unit of the detecting module 10 is arranged, attached or detached S10, at a fixed position on the subject's scalp 1.

Next, a first signal 3 generated in a specific state is measured S20 via the first unit 15.

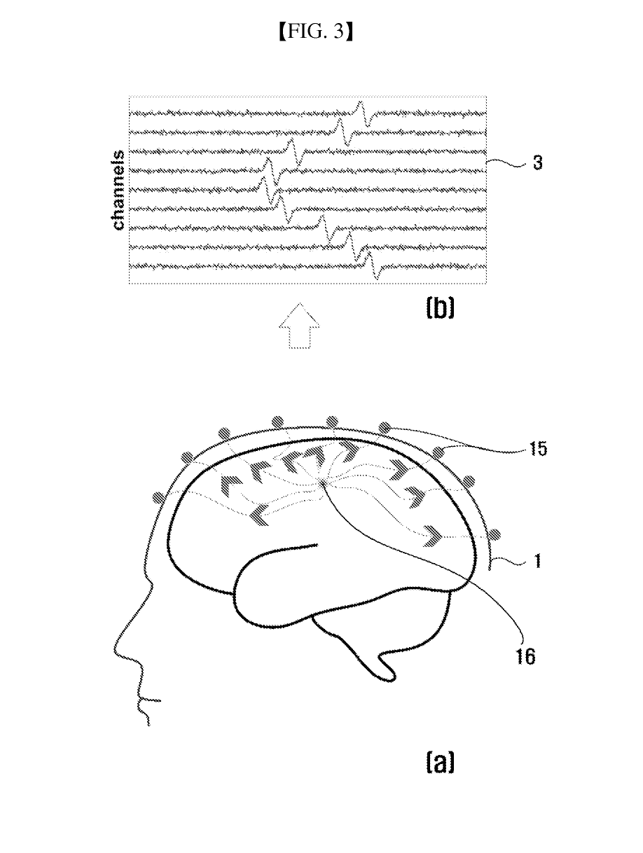

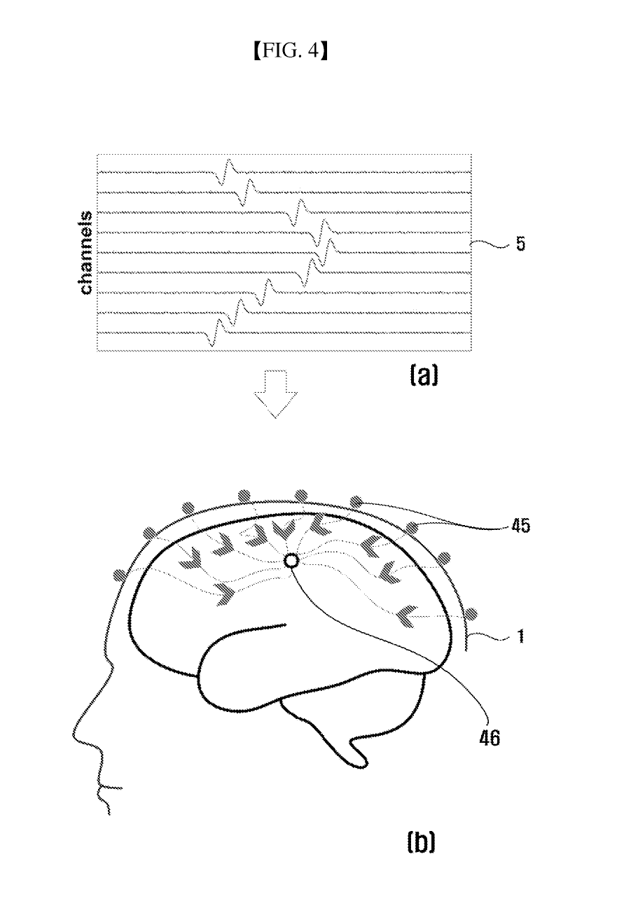

A second signal 5 (best seen in FIG. 4) transformed in a time-reversal order is generated from the first signal 3 (best seen in FIG. 3) via the controlling module 20 and the second signal 5 is stored S40 in the storage module 30.

After the second signal 5 is stored, subsequently, the first unit 15 measuring the first signal 3 is removed from the subject S50.

The step of signal measurement according to some embodiments is hereby complete.

As described before, the first signal 3 measured in some embodiments is directly stored without time-reversal transformation. Alternatively, the step of brain stimulation described below is subsequently performed without storing the first signal 3 or the second signal 5 transformed from the first signal 3.

The step of brain stimulation according to some embodiments includes touching one or more electrodes of the stimulating module 40 on the scalp. However, in some embodiments, one or more electrodes of the stimulating module 40 is/are in no contact with the scalp or is/are arranged to another particular position of the body of the subject.

In a case in which signals in a particular state measured at the subject's brain are stimulated, a second unit 45 is arranged, attached or detached S60, at the subject's brain, where the subject is the same as, or different from, the subject of which measuring and storing data are performed. The second unit 45 of the stimulating module 40 arranged to the subject is arranged at a position the same as, or similar to, the position of the first signal 3 to be restored in a particular state; in a case in which the subjects are different, positions of one or more stimulating modules 40 are different. If the positions of the first unit 15 and the second unit 45 in the same subject are the same, errors of stimulated positions due to inaccuracy of measuring positions occurred from transformation in a time-reversal order are diminished.

Next, the second signal 5 transformed in a time reversal order from the first signal 3 measured at a particular state and stored at a storage module 30 is provided, as shown in FIG. 4, to the second unit 45 via a controlling module 20, and the second signal 5 is sent to the brain S70 via the second unit 45. In this step, the controlling module 20, if the first signal 3 is directly stored in the storage module 30 or the controlling module 20 directly receives the first signal 3, in real time, transform the first signal 3 in a time reversal order to generate the second signal 5, and the controlling module 20 sends the second signal 5 to the second unit (45).

In some embodiments, the first signal 3 or the second signal 5 received from an external server or database other than the storage module 30 is sent, after being time reversed or directly, to the second unit 45.

Likewise, as the second signal 5 stimulates the subject's brain via the second unit 45, the same subject restores (or another subject reproduces) the specific state at a specific time premeasured by the second signal 5 via the brain, and is transited to the specific state, such as excitement, concentration, normality, and meditation. As such, by reproducing brain signals in a particular mode, emotional or psychological state is activated or deactivated.

Then, the second signal 5 is sent for a period of time, and the second unit 45 is removed from the subject S80.

The step of electroencephalogram stimulation and restoration of the method of brain signal management according to some embodiments is hereby complete.

In the meantime, although the some embodiments above illustrates a case in which only the first signal 3 of a particular state is measured and transformed into the second signal 5 via the controlling module 20 and is stored in the storage module 30, technical compositions of the some embodiments are not restricted to this. Thus, in some embodiments, the first signals 3 of a variety of certain mode is separately measured and stored in the storage module 30.

In other words, in some embodiments, one or more first signals 3 are measured in accordance with various states of the brain, such as a recognition enhanced state, emotion or stress controlling state, satiated state after eating, game playing state, awaken state, meditating state, excited state, or so. These first signals 3 are transformed via the controlling module 20 in a time reversal order to generate one or more second signals 5, and the one or more second signals 5 are released via the second unit 45 at the subject, thereby controlling each corresponding state.

As described thus far, according to some embodiments, by measuring a first signal which indicates a specific state of a brain, storing the first signal as the second signal transformed in a time reversal order and, in the case in which activation or deactivation of the brain of a specific state is to be performed, sending the second signal of a specific state to the brain; reproducing, improving, or controlling a certain psychological or brain mode become possible.

In concluding the detailed description, those skilled in the art will appreciate that many variations and modifications can be made to the preferred embodiments without substantially departing from the principles of the some embodiment described above. Therefore, the described preferred embodiments are used in a generic and descriptive sense only and not for purposes of limitation.

What is claimed is:

1. A brain signal management system, the system comprising:
a detecting module configured to measure a first signal which indicates a state of a brain; and
a controlling module configured to generate a second signal by transforming the first signal into the second signal in a time reversal order.

2. The system of claim 1, wherein the first signal is selected from the group consisting of electroencephalogram (EEG), near-infrared spectroscopy (NIRS), magnetic signal, and ultrasonic signal.

3. The system of claim 2, further comprising
a stimulating module configured to send the generated second signal to the brain or another brain.

4. The system of claim 3, wherein
the detecting module includes a first electrode configured to detect the first signal, and
the stimulating module includes a second electrode configured to send the generated second signal.

5. The system of claim 4, wherein the first electrode and the second electrode are included in one electrode.

6. The system of claim 1, further comprising a storage module configured to store at least one of the first signal and the second signal.

7. The system of claim 6, wherein the storage module is further configured to separately store one or more measured first signals according to a plurality of particular states of the brain, which are indicated by the measured first signals.

8. The system of claim 6, wherein the controlling module is further configured to
load the first signal stored in the storage module, and
generate the second signal based on the loaded first signal.

9. The system of claim 1, wherein the first signal and the second signal have different types from each other.

10. The system of claim 1, wherein the controlling module comprises an amplifying module configured to amplify the first signal.

11. The system of claim 1, wherein the controlling unit comprises a transform filter configured to
transform the first signal into an intermediate signal which is a time-reversed signal of the first signal, and
transform the intermediate signal into the second signal which has a different type from the first signal.

12. A brain signal management system, the system comprising:
a controlling module configured to
transform a first signal which indicates a state of a brain in a time reversal order, and
generate a second signal based on the transformed first signal; and
a stimulating module configured to send the generated second signal to the brain or another brain.

13. The system of claim 12, wherein the first signal is selected from the group consisting of electroencephalograms (EEG), near-infrared spectroscopy (NIRS), magnetic signals, and ultrasonic signals.

14. The system of claim 12, further comprising a storage module configured to store at least one of the first signal and the second signal.

15. The system of claim 14, wherein the storage module is further configured to separately store one or more measured first signals according to a plurality of particular states of the brain, which are indicated by the one or more measured first signals.

16. The system of claim 14, wherein the controlling module is further configured to
load the first signal stored in the storage module, and
transform the loaded first signal into the second signal.

17. The system of claim 12, wherein the first signal and the second signal have different types from each other.

18. The system of claim 12, wherein the controlling module comprises an amplifying module configured to amplify the first signal.

19. A method of brain signal management, performed by a brain signal management system, the method comprising:
measuring a first signal which indicates a state of a brain; and
generating a second signal by transforming the first signal in a time reversal order into the second signal.

20. The method of claim 19, further comprising sending the generated second signal to the brain or another brain.
